(19) United States
(12) Patent Application Publication
(10) Pub. No.: US 2023/0016621 A1
(43) Pub. Date: Jan. 19, 2023

(54) Systems and Methods for Targeted Thrombolytic Delivery

(71) Applicant: Bard Access Systems, Inc., Salt Lake City, UT (US)

(72) Inventors: Steffan Sowards, Salt Lake City, UT (US); William Robert McLaughlin, Bountiful, UT (US); Devan Anderson, Taylorsville, UT (US); Anthony K. Misener, Bountiful, UT (US)

(21) Appl. No.: 17/865,146

(22) Filed: Jul. 14, 2022

Related U.S. Application Data
(60) Provisional application No. 63/222,342, filed on Jul. 15, 2021.

(51) Int. Cl. A61M 25/00

(52) U.S. Cl. CPC A61M 25/00; A61M 2025/0019; A61M 2210/12

(57) ABSTRACT

Devices and methods for restoring patency of a catheter. Devices can include an elongate member configured for insertion through the catheter. Elongate members can facilitate delivery of a chemical agent directly to a catheter blockage and/or facilitate agitation of the chemical agent in close proximity to the blockage. A catheter or catheter system disclosed herein includes a device for removing the blockage. The device for removing a blockage from a catheter can include an elongate member configured for insertion through a catheter lumen, the elongate member defining a proximal end and a distal end, and an agitation actuator coupled with the elongate member adjacent the proximal end, wherein operation of the actuator causes agitation of a fluid adjacent the distal end.

### PRIORITY

This application claims the benefit of priority to U.S. Provisional Application No. 63/222,342, filed Jul. 15, 2021, which is incorporated by reference in its entirety into this application.

### BACKGROUND

Long term catheters can be subject to occlusions or blockages that reduce or destroy patency of the catheter. For example, it has been reported that occlusion of central venous catheters occurs in 14 to 36 percent of patients within 1-2 years of catheter placement. Occlusion can arise from mechanical obstruction, precipitation of medications or parenteral nutrition, or thrombotic causes. Catheters can also become occluded due to formation of a fibrin sheath around the catheter tip, where formation of a fibrin sheath is reported to be one of the most common causes of thrombotic obstruction. Intraluminal clots account for 5-25% of all catheter occlusions and may cause complete catheter obstruction.

A common practice is to treat suspected thrombotic occlusions with thrombolytic agents. Current recommendations include administration of a thrombolytic agent into the catheter lumen with a dwell time of at least 30 minutes and a repeated dose if needed. Dwell times up 120 minutes are not un-common.

The systems, devices, and methods described herein may aid in the removal of a catheter blockage and restore catheter patency. More specifically, the systems, devices, and methods may aid in dispensing a thrombolytic agent at the blockage location, aid in penetrating the blockage, prevent loss of the thrombolytic agent into the blood stream, and/or increase a surface area of the blockage in contact with the thrombolytic agent.

### SUMMARY

Briefly summarized, disclosed herein is an instrument for removing a blockage from a catheter, such as an intravenous catheter. The instrument includes an elongate member configured for insertion in a catheter lumen having the elongate member defining a proximal end and a distal end and an agitation actuator coupled with the elongate member adjacent the proximal end. The agitation actuator causes agitation of a fluid adjacent the distal end of the elongate member.

The distal end of the elongate member may be disposed within the lumen and the fluid may be disposed adjacent the blockage of the catheter. The fluid may include a chemical agent, including thrombolytic agent, configured to decompose the blockage. The blockage may be disposed within the lumen, and the agitation of the fluid may occur within the lumen. In some embodiments, the blockage extends beyond a distal tip of the catheter, such as when the blockage includes a fibrin sheath, and the blockage may fully occlude the catheter.

The agitation actuator may be an electro-mechanical device and the agitation may include a longitudinal and/or lateral vibration of the distal end of the elongate member. In some embodiments, the vibration may include an ultrasound frequency. The elongate member may include one or more protrusions extending away from the elongate member at the distal end.

Also disclosed herein is a device for removing a blockage from a catheter which may be an intravenous catheter. The device includes a pressure actuator coupled with the catheter at a proximal end of the catheter so that the pressure actuator is in fluid communication with the catheter lumen. The pressure actuator produces a fluctuating fluid pressure along the lumen to cause agitation of a fluid adjacent the blockage of the catheter. The fluid includes a chemical agent configured to decompose the blockage, and the chemical agent may include thrombolytic agent. The agitation of the fluid may occur within the lumen.

The pressure actuator may be an electro-mechanical device. Operation of the pressure actuator may define a positive pressure and/or a negative pressure within the lumen. Operation may also cause pressure fluctuations within the lumen between a positive pressure and a negative pressure.

Also disclosed herein is a catheter, which may be an intravenous catheter. The catheter includes an outer elongate tubular member defining an outer lumen extending between a proximal end and a distal end of the outer member and an inner elongate tubular member defining an inner lumen extending between a proximal end and a distal end of the inner member. The inner member includes a plurality of side ports disposed along a ported portion of the inner member, where the ports are in fluid communication with the inner lumen. The inner member is disposed within the outer lumen, and the inner member is displaceable with respect to the outer member between a distal position and a proximal position. When the inner member is in the proximal position, the ports are covered by the outer member, and when the inner member is in the distal position, the ports are exposed. The inner member may be continuously positionable between the distal position and a proximal position, and the inner member may be removable from the outer member. The inner lumen may also be open at the distal end and fluid may be exchanged with a patient through the inner lumen.

In use, a chemical agent may be dispensed through the inner lumen toward a blockage of the catheter to contact the blockage. The fluid may include a chemical agent configured to decompose the blockage, and the chemical agent may include thrombolytic agent. In further use, the inner member is distally displaced with respect to the outer member to expose the side ports, and the chemical agent may be dispensed through the side ports to contact the blockage outside of the inner member. the chemical agent may also contact a blockage extending beyond the distal end of the inner member and/or extending proximally along an outside surface of the inner member. The chemical agent dispensed through the side ports may also contact the blockage along an outside surface of the blockage.

Also disclosed herein is another instrument for removing a blockage from a catheter which may be an intravenous catheter. The instrument includes an elongate member configured for insertion through a catheter lumen, the elongate member defining a proximal end and a distal end. An inflatable balloon is coupled with the elongate member adjacent the distal end, and the balloon is in fluid communication with a first lumen extending between the balloon and the proximal end of the elongate member. A plurality of side ports is disposed along a ported portion of the elongate member, and the side ports are in fluid communication with a second lumen extending between side ports and the proximal end of the elongate member.

The ported portion is disposed proximal the balloon. The distal end of the elongate member is configured to pierce and extend through a blockage of the catheter so that, in use (i) the blockage is disposed between a proximal end and a distal end of the ported portion, (ii) one or more side ports are disposed distal the blockage, and/or (iii) one or more sides ports are disposed proximal the blockage. In some embodiments, the balloon extends across the catheter lumen when inflated.

In use, the elongate member may be coupled with a first fluid device at the proximal end so that the first fluid device is in fluid communication with the first lumen, and the first fluid device is configured to inflate and deflate the balloon.

In use, the elongate member may be coupled with a second fluid device at the proximal end so that the second fluid device is in fluid communication with the second lumen, and the second fluid device is configured to dispense a chemical agent through the second lumen. In use, the chemical agent may be dispensed through the side ports to contact with the blockage along the ported portion including the proximal and distal end portions of the blockage. In use, the second fluid device may be manipulated to withdraw and re-dispense the chemical agent through the side ports to agitate the chemical agent, and the instrument is longitudinally displaced within the lumen to agitate the chemical agent.

In use, the instrument may be proximally displaced within the lumen to contact the blockage with the balloon and/or proximally displace one or more portions of the blockage along the catheter lumen.

Also disclosed here is a method of restoring patency of a catheter. The method includes dispensing a chemical agent through a catheter lumen, the chemical agent configured to decompose a blockage of the lumen and agitating the chemical agent to enhance decomposition of the blockage.

Agitating the chemical agent may include inserting an elongate member within the catheter lumen and may include displacing a distal end of the elongate member to cause agitation. The agitating may further include vibrating of the distal end of the elongate member.

In some embodiments of the method, the elongate member includes a member lumen, and dispensing a chemical agent may include dispensing the chemical agent through the member lumen. The elongate member may include a plurality of side ports, and dispensing a chemical agent may include dispensing the chemical agent through the side ports. Dispensing the chemical agent through the side ports may also include dispensing chemical agent on a proximal side and a distal side of the blockage.

In some embodiments of the method, inserting an elongate member may include piercing the blockage with a distal tip of the elongate member. In some embodiments, agitating the chemical agent includes fluctuating a pressure of the chemical agent.

Also disclosed herein is a catheter system. The catheter system includes a catheter defining a catheter lumen extending between a proximal end and a distal end and an elongate member configured for insertion through the catheter lumen, where the elongate member defines a proximal member end and a distal member end. The elongate member is configured to perform one or more operations to remove a blockage of the catheter.

The operations may include agitating a chemical agent disposed in contact with the blockage, where agitating the chemical agent includes displacing the distal member end and/or vibrating the distal member end.

The operations may include dispensing the chemical agent through a first member lumen of the elongate member. In some embodiments, the elongate member includes a plurality of side ports extending along a ported portion of the elongate member, and the operations include dispensing the chemical agent through the side ports. The operations may further include dispensing the chemical agent on a distal side of the blockage.

In some embodiments of the method, the elongate member includes an inflatable balloon at the distal end; and the operations include inflating the balloon on a distal side of the blockage.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a side cross-sectional view of a first embodiment of medical instrument for use in removing a catheter blockage, in accordance with some embodiments;

FIG. 2 illustrates a side cross-sectional view of a second embodiment of medical instrument for use in removing a catheter blockage, in accordance with some embodiments;

FIG. 3 illustrates a side cross-sectional view of a catheter including blockage removal features, in accordance with some embodiments;

FIG. 4A illustrates a side cross-sectional view of a third embodiment of medical instrument for use in removing a catheter blockage, in accordance with some embodiments; and

FIG. 4B is a detailed cross-sectional view of a portion of medical instrument of FIG. 4A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Unless defined otherwise, all technical and scientific terms used herein have the same meaning as commonly understood by those of ordinary skill in the art.

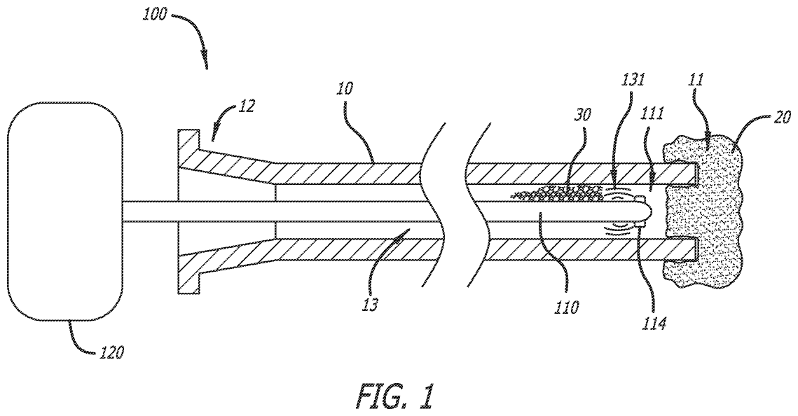

FIG. 1 illustrates a first exemplary embodiment of a medical instrument 100 for removing a blockage 20 obstructing a catheter 10. The instrument 100 includes an elongate shaft 110 configured for (i) insertion through a lumen 13 of the catheter 10 and (ii) extension between a proximal end 12 and a distal end 11 of the catheter 10. The catheter 10 may be an intravenous catheter.

The blockage 20 may define a partial occlusion or an entire occlusion of the catheter lumen 13. In some embodiments, the blockage 20 or a portion thereof may be disposed within the catheter lumen 13. In other embodiments, the blockage 20 may cover the distal end 11. In some embodiments, the blockage 20 may extend proximally along an exterior of the catheter 10, where the blockage 20 may include a fibrin sheath, for example. The blockage 20 may comprise a fluid precipitate or result from the formation of a thrombus within the lumen 13 or adjacent the distal end 11.

The instrument 100 is configured to define an agitation 131 of a chemical agent 30 disposed within the lumen 13 adjacent the blockage 20. The shaft 110 is coupled with an agitation actuator 120 (actuator) at a proximal end 112 of the shaft 110. The actuator 120 causes agitating movement of the distal end 111 of the shaft 110. The agitation may comprise oscillating movement of the distal end 111 in a longitudinal and/or later direction. In some embodiments, the agitation may comprise a rotation of the shaft 110. The shaft 110 may include one or more protrusions 114 to aid in the agitation 131 of the chemical agent 30. The protrusions 114 may include posts, fins, flaps or any protrusion suitably shaped to aid in the agitation 131 when the distal end 111 is displaced.

The actuator 120 may be an electro-mechanical device. In some embodiments, the agitating movement may include a vibration of the shaft 110 and the vibration may include frequency within the ultrasound range (i.e., a frequency above about 20 kilohertz). In some embodiments, the actuator 120 may be configured for manual operation. As such, the actuator 120 may include a plunger, a crank, or any other suitable physical interface to facilitate a manually defined agitating movement of the shaft 110 by a clinician.

The chemical agent 30 may be configured to dissolve, decompose or otherwise breakdown the solid nature of the blockage 20. As such, decomposition of the blockage 20 may be facilitated by a chemical reaction of the chemical agent 30 adjacent a surface the blockage 20. The rate of the chemical reaction (i.e., rate of decomposition) may be limited by a diffusion rate of the chemical agent 30 with respect to the blockage 20. In some instances, the rate of decomposition may decrease over time due to a decrease in the diffusion of the chemical agent 30 adjacent the surface the blockage 20. As such, agitation of the chemical agent 30, adjacent the surface the blockage 20, may restore, maintain, or otherwise enhance the diffusion rate of chemical agent 30 adjacent the surface the blockage 20, thereby enhancing the rate of decomposition of the blockage 20. By way of summary, the agitation 131 of the chemical agent 30 adjacent the blockage 20 may reduce the time necessary to remove the blockage 20 and restore patency of the catheter 10. In some embodiments, the agitation 131 may physically breakdown (e.g., fragment) the blockage 20 further enhancing the decomposition process.

In use, the clinician may dispense the chemical agent 30 to the blockage 20 through the lumen 13 of the catheter 10. The clinician may then insert the instrument 100 (or more specifically the shaft 110) into the lumen 13 of the catheter 10 and advance the shaft 110 so that the distal end 111 of the shaft 110 is disposed adjacent the blockage 20. The clinician may then operate the actuator 120 to cause the agitation 131 until the blockage 20 is adequately decomposed. The clinician may then withdraw the shaft 110 from the catheter 10.

In some embodiments, the distal end 111 of the shaft 110 includes the chemical agent 30 deposited thereon (e.g., in a powder or gel form). In embodiments in which the chemical agent 30 is deposited on the distal end 111 in a powder form, the powder may mix with and/or dissolve in fluid dispensed into the lumen 13.

The instrument 100 may define a stand-alone device for use with the catheter 10. In other embodiments, the instrument 100 or one or more components thereof may be combined with the catheter 10 to define a catheter system.

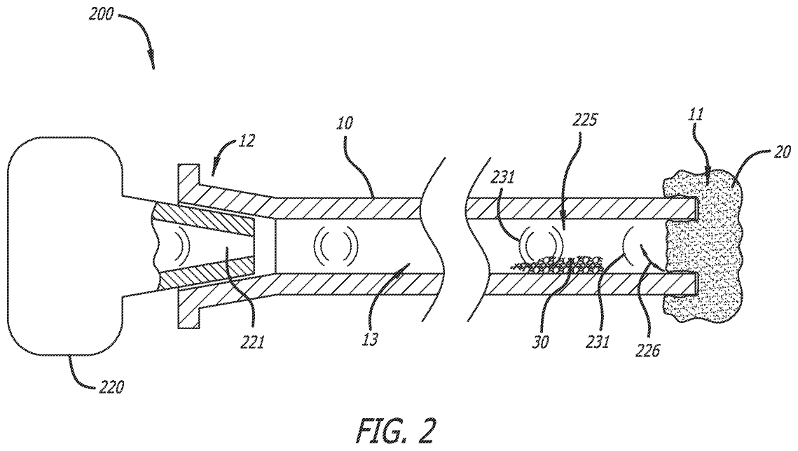

FIG. 2 illustrates a second exemplary embodiment of a medical instrument (or device) 200 for removing the blockage 20 from the catheter 10. The instrument 200 includes a pressure actuator 220 (e.g., a hydraulic pressure driver) coupleable to the catheter 10 at a proximal end 12 of the catheter 10 via a pressure port 221.

The pressure actuator 220 is configured to define a fluctuating/oscillating pressure 225 along the lumen 13 of the catheter 10. The fluctuating pressure 225 causes agitation 231 of the chemical agent 30 adjacent the blockage 20. The pressure actuator 220 may also be configured to displace and/or dispense fluid such as the chemical agent 30 distally along the catheter lumen 13 to deliver the chemical agent 30 adjacent the blockage 20.

In some embodiments, the pressure actuator 220 may be configured for manual operation by the clinician to generate the pressure fluctuations. As such, the pressure actuator 220 may comprise a plunger, a crank, or any other suitable physical interface to enable the clinician to manually generate the pressure fluctuations 225. For example, the pressure actuator 220 may be a syringe, and manual operation may include depressing and/or withdrawing the plunger of the syringe. In other embodiments, the actuator 220 may be an electro-mechanical device such as a reciprocating plunger within cylinder, for example. The pressure fluctuations 225 may occur across (i) a positive pressure range (i.e., a pressure range above atmospheric pressure), (ii) a negative pressure range (i.e., a pressure range below atmospheric pressure), or (iii) both the positive pressure range and the negative pressure range.

Similar to the agitation 131 described above, the agitation 231 of the chemical agent 30 adjacent the surface the blockage 20 may restore, maintain, or otherwise enhance the rate of decomposition. By way of summary, the agitation 231 of the chemical agent 30 adjacent the blockage 20 may reduce the time necessary to remove the blockage 20 and restore patency of the catheter 10. In some embodiments, the agitation 231 may induce localized fluid currents 226 adjacent a surface of the blockage 20 to physically breakdown (e.g., fragment) the blockage 20, thereby further enhancing the decomposition process.

In use, the clinician may dispense the chemical agent 30 to the blockage 20 through the lumen 13 of the catheter 10. The clinician may then couple the pressure actuator 220 to the catheter 10 at the proximal end 12. The clinician may then activate the pressure actuator 220 to cause the agitation 231 until the blockage 20 is adequately decomposed. The clinician may then decouple the pressure actuator 220 from the catheter 10.

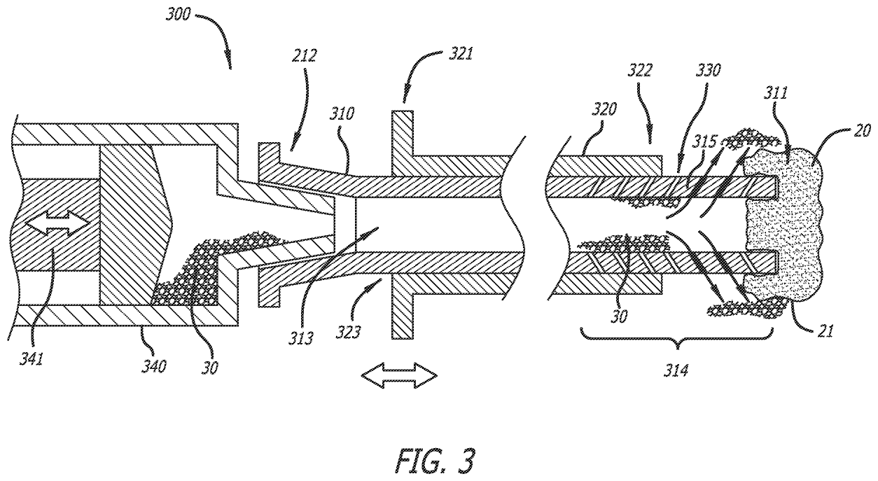

FIG. 3 illustrates an exemplary embodiment of a catheter 300 including features and functionalities for removing the blockage 20 of the catheter 300. The catheter 300 includes an inner tubular member 310 defining a lumen 313 extending between a distal end 311 and proximal end 312. The catheter 300 further includes an outer tubular member 320 (e.g., an outer catheter) defining an outer lumen 323 extending between a distal end 321 and proximal end 322 of the outer member 320. The inner member 310 is slidably disposed within the lumen 323 of the outer member 320 so that the outer member 320 is continually displaceable between a distal position and a proximal position with respect to the inner tubular member 310.

In some embodiments, the outer member 320 may be configured to function as a stand-alone catheter. In other words, the outer member 320 may be a catheter such as a central venous catheter, for example. In such embodiments, the inner tubular member 310 may be a separate device to be inserted into the outer tubular member.

The inner member 310 comprises a plurality of side ports (or channels) 330 extending along a ported portion 314 of the inner member 310. The sides ports 330 extend through a tubular wall 315 of the inner member 310. The ported portion 314 is positioned with respect to the outer member 320 such that (i) when the outer member 320 is disposed in the distal position, the distal end of the 322 is extend distally beyond the ported portion 314 so that the side ports are closed/occluded by the outer member 320 and (ii) when the outer member 320 is disposed in the proximal position, the distal end of the 322 is positioned proximal the ported portion 314 so that the side ports 330 are open (i.e., uncovered by the outer member 320. By way of summary, the outer member 320 may occlude all of the side ports 330 when disposed in the distal position. As the outer member 320 is retracted from the distal position, a greater number of side ports 330 are uncovered/opened until the outer member 320 is disposed in the proximal position at which position, all of the side ports 330 may be uncovered/opened. Alternatively, the inner member 310 may be continually inserted such that the side ports 330 are uncovered/opened.

In use, the chemical agent 30 dispensed through the side ports 330 may contact the blockage at multiple locations of the blockage 20. In some instances, the blockage 20 may extend proximally along an exterior of the catheter 300 including outer member 320 and/or the inner member 310. In such instances, the chemical agent 30 dispensed through the side ports 330 may be directed proximally along an exterior of the catheter 300 and contact the portions of the blockage 20 disposed along proximally along the exterior of the catheter 300. In some embodiments, the blockage 20 may extend proximally along the ported portion 314. As such, the chemical agent 30 dispensed through the side ports 330 may contact the blockage 20 along an annular inside surface of the blockage 20.

In instances of the blockage 20 of the catheter 300, the catheter 300 (more specifically the inner tubular member 310) may be coupled with a fluid device (e.g., a syringe 340 having a plunger 341) at the proximal end 312 so that the syringe 340 is in fluid communication with the lumen 313 and the syringe 340 may contain the chemical agent 30. In such instances, the clinician may depress the plunger 341 to dispense the chemical agent 30 through the lumen 313 to the blockage 20. If the outer tubular member 320 is displaced away from the distal position, the chemical agent 30 may flow out of the lumen 313 through the opened side ports 330 to expose an outside surface 21 of the blockage 20 to the chemical agent 30. Exposing the outside surface 21 may enhance decomposition of the blockage 20.

In some instances, the clinician may retract the plunger to proximally displace the chemical agent 30 within the lumen 313. In further instances, the clinician may repeatedly depress and retracted to the plunger 341 to agitate the chemical agent 30 adjacent the blockage 20. The agitation may further enhance the rate of decomposition.

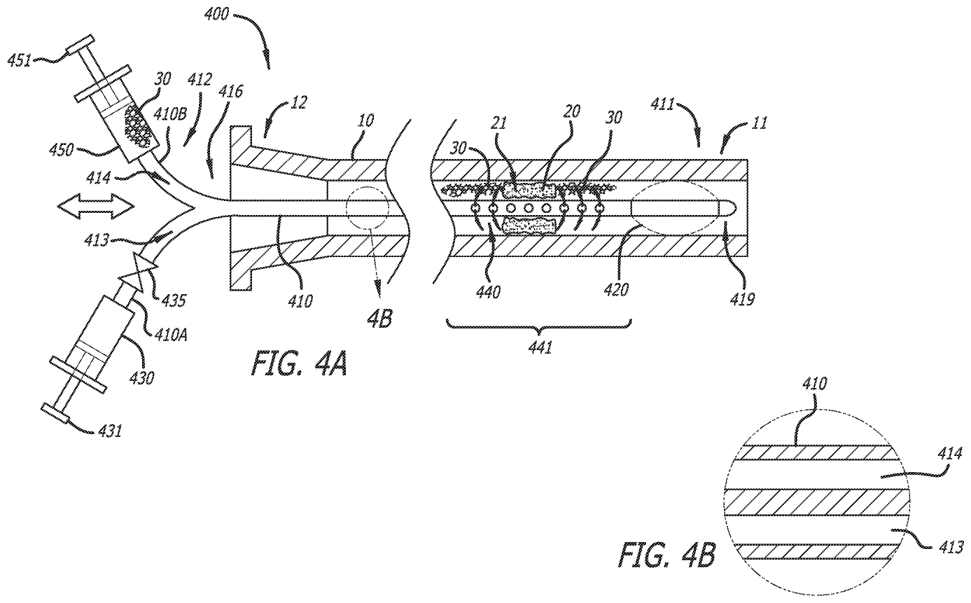

FIG. 4A illustrates a third exemplary embodiment of a medical instrument 400 for removing the blockage 20 of the catheter 10. The instrument 400 includes an elongate bi-luminal tubular member 410 configured for (i) insertion through the lumen 13 of the catheter 10 and (ii) extension between the proximal end 12 and the distal end 11 of the catheter 10. The catheter 10 may be an intravenous catheter. The instrument 400 is configured to deliver the chemical agent 30 to the blockage 20 within the lumen 13. More specifically the instrument 400 is configured to deliver the chemical agent 30 to both the proximal side and the distal side of the blockage 20.

FIG. 4B is a detailed cross-sectional view of a portion of the tubular member 410. Referring to FIGS. 4A and 4B, the tubular member 410 defines a distal end 411 and a proximal end 412. A first lumen 413 and a second lumen 414 extend along the tubular member 410. The tubular member 410 includes a bi-furcation 416 adjacent the proximal end 412 to define a first leg 410A and a second leg 410B. The first lumen 413 extends along the first leg 410A and the second lumen 414 extends along the second leg 410B. A distal tip 419 of the tubular member 410 is configured for piercing the blockage 20 so that the tubular member 410 may be extended through an opening 21 of the blockage 20.

The instrument 400 includes an inflatable balloon 420 coupled with the tubular member 410 adjacent the distal end 411. The instrument 400 may also include a first fluid device, for example, a first syringe 430 including a first plunger 431, coupled with the first leg 410A. The balloon 420 is fluidly coupled with the first fluid device 430 via the first lumen 413. Operation of the syringe 430 causes inflation and deflation of the balloon 420. In an inflated state, the balloon 420 may extend across the catheter lumen 13 and in some embodiments fill the cross section of the catheter lumen 13. A valve 435 may be disposed in line with the first lumen 413 along the first leg 410A to selectively allow and prevent fluid flow through the lumen 413.

A plurality of side ports 440 extend along a ported portion 441 of the tubular member 410 proximal the balloon 410. A second fluid device, for example, a second syringe 450 including a second plunger 451, may be coupled with the second leg 410B. The side ports 440 are each fluidly coupled with the second syringe 450 via the second lumen 414. Operation of the second syringe 430 causes fluid flow through the ports 440. In some embodiments, the second syringe 450 may contain the chemical agent 30, and depressing the second plunger 451 may dispense the chemical agent 30 through the side ports 440. As the side ports 440 are disposed proximal the balloon 420 and as the balloon 420 may extend across the catheter lumen 420, the balloon 420 may inhibit loss of the chemical agent 30 into the blood stream of the patient.

By way of use, in the event of the blockage 20 of the catheter lumen 13, the clinician may insert the instrument 400 (or more specifically the tubular member 410) into the lumen 13 of the catheter 10 and advance the tubular member 410 so that the distal end 411 of the tubular member 410 is disposed adjacent the blockage 20. The clinician may then further advance to the tubular member 410 to pierce the blockage 20 with the distal tip 419 and advance the tubular member 410 through the blockage 20. The clinician may position the tubular member 410 so that the balloon 420 is distal the blockage 20. The clinician may further position the tubular member 410 so that blockage 20 is disposed along the ported portion 441, i.e., between a proximal end and a distal end of the ported portion. The clinician may depress the first plunger 431 to inflate the balloon 420 so that the balloon 420 extends across the catheter lumen 13. With the balloon inflated, the clinician may close the valve 435 to lock the inflation pressure within the balloon 420. The clinician may then depress the second plunger 451 to dispense the chemical agent 30 through the side ports 440. The dispensed chemical agent 30 may contact the blockage 20 on the proximal side and/or the distal side of the blockage 20. The chemical agent 30 may also contact the blockage 20 along the opening 21. After adequate decomposition of the blockage 20, the clinician may open the valve 435 and retract the first plunger 431 to deflate the balloon 20. The clinician may extract the tubular member 410 from the catheter lumen 13.

In some embodiments, the clinician may retract the second plunger 451 to withdraw the chemical agent 30 through the side ports 440. The clinician may also repeatedly depress and retracted to the second plunger 451 to agitate the chemical agent 30 adjacent the blockage 20. In further embodiments, the clinician may longitudinally displace the tubular member 410 along the catheter lumen 13 to agitate the chemical agent 30. The agitation may further enhance the rate of decomposition. In some instances, the clinician may proximally displace the tubular member 410 with the balloon 420 in the inflated state to drag any remaining fragments or portions of the blockage 20 out of the catheter lumen 13.

The instrument 400 may define a stand-alone device for use with the catheter 10. In other embodiments, the instrument 400 or one or more components thereof may be combined with the catheter 10 to define a catheter system.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An instrument for removing a blockage from a catheter lumen, comprising:
an elongate member configured for insertion in the catheter lumen; and
an agitation actuator coupled with the elongate member adjacent a proximal end thereof, wherein operation of the agitation actuator causes agitation of a fluid adjacent the distal end of the elongate member.

2. The instrument according to claim 1, wherein the fluid includes a chemical agent configured to decompose the blockage and is disposed adjacent the blockage of the catheter.

3. The instrument according to claim 2, wherein the chemical agent includes thrombolytic agent.

4. The instrument according to claim 1, wherein the blockage extends beyond a distal end of the catheter lumen.

5. The instrument according to claim 4, wherein the blockage includes a portion of a fibrin sheath.

6. The instrument according to claim 1, wherein the agitation comprises a longitudinal or lateral displacement of the distal end of the elongate member.

7. The instrument according to claim 1, wherein the agitation comprises a rotation of the distal end of the elongate member.

8. The instrument according to claim 1, wherein the agitation actuator is an electro-mechanical device.

9. The instrument according to claim 8, wherein the agitation comprises a longitudinal vibration of the distal end.

10. The instrument according to claim 8, wherein the agitation comprises a lateral vibration of the distal end.

11. The instrument according to claim 9, wherein the vibration includes an ultrasound frequency.

12. The instrument according to claim 1, wherein the elongate member comprises one or more protrusions extending away from the elongate member at the distal end thereof.

13. The instrument according to claim 1, wherein the catheter is an intravenous catheter.

14. A device for removing a blockage from a catheter, comprising:
a pressure actuator coupled with the catheter at a proximal end thereof, the pressure actuator in fluid communication with a catheter lumen,
wherein the pressure actuator is configured to produce a fluctuating fluid pressure along the lumen to cause agitation of a fluid adjacent a blockage of the catheter.

15. The device according to claim 14, wherein the catheter is an intravenous catheter.

16. The device according to claim 14, wherein the fluid includes a chemical agent configured to decompose the blockage.

17. The device according to claim 16, wherein the chemical agent includes a thrombolytic agent.

18. The device according to claim 14, wherein the agitation of the fluid occurs within the catheter lumen.

19. The device according to claim 14, wherein operation of the pressure actuator defines a positive pressure and/or a negative pressure within the catheter lumen.

20. The device according to claim 19, wherein operation of the pressure actuator defines pressure fluctuations within the lumen between the positive pressure and the negative pressure.

21. The device according to claim 14, wherein the pressure actuator is an electro-mechanical device.

22-39. (canceled)
